(19) United States
(12) Patent Application Publication
(10) Pub. No.: US 2024/0350090 A1
(43) Pub. Date: Oct. 24, 2024

(54) PIVOTABLE PATIENT TABLE FOR A MEDICAL IMAGING APPARATUS AND SYSTEM HAVING PATIENT TABLE AND IMAGING APPARATUS

(71) Applicant: Siemens Healthineers AG, Forchheim (DE)

(72) Inventors: Arne Hengerer, Möhrendorf (DE); Benjamin Hofmann, Großenseebach (DE); Bernhard Krauss, Burgthann (DE); Martino Leghissa, Wiesenthau (DE)

(21) Appl. No.: 18/644,080

(22) Filed: Apr. 23, 2024

(30) Foreign Application Priority Data
Apr. 24, 2023 (DE) 10 2023 203 735.1

(51) Int. Cl. A61B 5/00; A61B 5/055

(52) U.S. Cl. CPC A61B 5/704; A61B 5/055

(57) ABSTRACT

A patient table for a medical imaging apparatus, and a system including a patient table and an imaging apparatus are provided. The patient table has a pivoting apparatus that is configured to pivot a patient about a vertical axis in an examination position.

This application claims the benefit of German Patent Application No. DE 10 2023 203 735.1, filed Apr. 24, 2023, which is hereby incorporated by reference in its entirety.

### BACKGROUND

The present embodiments relate to a patient table for a medical imaging apparatus, and a system including a patient table and an imaging apparatus. The patient table has a pivoting apparatus that is configured to pivot a patient about a vertical axis in the examination position.

Independent of the grammatical term usage, individuals with male, female or other gender identities are included within the term.

Medical imaging apparatuses relate to apparatuses in which the patient is introduced into the apparatus for imaging. For this purpose, the patient is supported on a patient table and is moved into the imaging apparatus with the patient table. Examples of such imaging apparatuses are magnetic resonance tomographs or X-ray computed tomographs having an opening into which the patient is introduced or moved through for image acquisition.

Such imaging apparatuses are also used for monitoring interventions (e.g., biopsies, interventions with catheters, or in minimally invasive interventions). In this case, the operating physician also needs access to the patient (e.g., in the area in which the intervention takes place, but that is also to be imaged at the same time).

Magnetic resonance tomographs are apparatuses that image an object to be examined or a patient by orienting nuclear spins of the object to be examined with a strong external magnetic field and by exciting an alternating magnetic field for precession about this orientation. The precession or return of the spins from this excited state into a state with lower energy generates an alternating magnetic field in response. The alternating magnetic field is received via antennas.

With the aid of magnetic gradient fields, a spatial coding is impressed on the signals for imaging, which subsequently makes it possible to allocate the received signal to a volume element. The received signal is then evaluated, and a three-dimensional imaging representation of the object to be examined is provided.

The magnetic field is generated using a superconducting magnet that surrounds the patient. The patient is located in the patient tunnel. The patient tunnel extends through the magnet. In a center of the patient tunnel (e.g., the field of view or iso-center), the required homogeneous magnetic field is provided. In the past, the patient tunnel was kept as small as possible in order to limit the structural requirements for the magnet.

With the new developments in signal acquisition and evaluation, it is possible to realize comparable image quality and signal-to-noise ratios (SNR) with weaker magnets, which also allow patient tunnels with a larger opening width.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, interventions are facilitated by an operating physician in an imaging apparatus.

The patient table in accordance with the present embodiments is provided for a medical imaging apparatus. The patient table is configured to support a patient in an examination position in the imaging apparatus for imaging. For example, the patient table is configured to introduce the patient into a patient tunnel (e.g., in a magnetic resonance tomograph). The patient table may have a bed surface for the patient that is oriented essentially horizontally. The patient table may further include a positioning unit that is configured to move the bed surface into the patient tunnel. The positioning unit is arranged outside the patient tunnel in order to save space inside the patient tunnel and to reduce interference in the imaging on account of motor units.

The imaging apparatus may be a magnetic resonance tomograph, but use with a computed tomograph may also be provided, for example.

The patient table has a pivoting apparatus that is configured to pivot or to rotate the patient about a vertical axis in an examination position for an acquisition of an image. In this case, an axis that is oriented essentially parallel to a direction of gravity or vertically is considered to be a vertical axis. The axis is not necessarily a physically present axis that is part of the pivoting apparatus, but, for example, a virtual geometric axis that defines the reference point for the rotation or pivoting movement.

The vertical axis may be arranged with respect to the patient table so that, in the case of imaging, the vertical axis extends through the imaging apparatus (e.g., an image acquisition region of the imaging apparatus). In this case, the vertical axis may extend essentially through an isocenter or center of symmetry of the image acquisition region, so that the same region of the patient is still imaged during a pivoting movement, only rotated by an angle.

Embodiments of the pivoting apparatus are explained in more detail with reference to the following.

The pivoting apparatus may make it possible to change an orientation of the patient table in the vicinity of the image acquisition region, so that access for an operating physician to the image acquisition region and the patient is facilitated.

The system in accordance with the present embodiments has a patient table for a medical imaging apparatus (e.g., a magnetic resonance tomograph).

The patient table is configured to support a patient in an examination position in the imaging apparatus for imaging, and, for example, to retract into a patient tunnel in a magnetic resonance tomograph.

The system also has a support apparatus. A support apparatus is an apparatus that is used to arrange the patient table in a predetermined and variable position relative to the imaging apparatus. In one embodiment, the support apparatus may be arranged on the imaging apparatus. The support apparatus may also be, for example, an installation on the floor or the ceiling of a room in which the imaging apparatus is located. The support apparatus may also be a floor or a ceiling of a room in which the imaging apparatus is located.

The system in accordance with the present embodiments has a pivoting apparatus that is configured to pivot the patient about a vertical axis in the examination position. The statements already made in connection with the patient table in accordance with the present embodiments apply to the vertical axis. The patient table of the system in accordance with the present embodiments differs from the patient table in accordance with the present embodiments described above in that at least parts of the pivoting apparatus are provided not only by the patient table, but also by the support apparatus. Various embodiments of how the system provides the pivoting apparatus are illustrated below.

The system in accordance with the present embodiments shares the advantages of the patient table in accordance with the present embodiments.

Further embodiments are explained below.

In one possible embodiment of the patient table, the patient table has a suspension apparatus for fastening to a support apparatus. The suspension apparatus is configured to arrange the patient table in a suspended manner (e.g., without a direct connection to the floor) in a room. The suspension apparatus may be connected to a support apparatus in order to arrange the patient table at a predetermined location in a room and/or relative to an imaging apparatus. For this purpose, the support apparatus may be, for example, a simple ceiling fastening or a self-supporting construction that extends above the imaging apparatus.

The fastening of the patient table via the suspension apparatus makes it possible to keep the floor and an area below the patient table clear. This thus improves access to the patient in the imaging apparatus.

In one embodiment of the patient table, the pivoting apparatus is part of the suspension apparatus. For example, a vertical axis or pivot joint, about which the patient table may be rotated or pivoted, may be provided in the suspension apparatus.

In the suspension apparatus, a pivoting apparatus may be realized in a simple and space-saving manner.

In one possible embodiment of the patient table, the pivoting apparatus has a pivoting arm. The pivoting arm is configured to guide the patient table during a pivoting movement about the vertical axis. For this purpose, the pivoting arm may have a joint, hinge, or other rotatable apparatus at the location of the vertical axis, which renders possible a rotational movement of the pivoting arm about the vertical axis. At the location of the vertical axis, a corresponding pivot joint may be provided. The pivoting arm produces a rigid connection between the patient table and the vertical axis, so that the distance and the orientation toward the vertical axis remain unchanged during the pivoting movement.

However, a pivoting arm may also be provided. A first pivot joint of the pivoting arm is arranged under the patient table outside or in front of the patient tunnel. The pivoting apparatus may have further elements in order to restrict the pivoting movement such that the axis of rotation of the pivoting movement nevertheless remains as close as possible to the isocenter. For example, the pivoting arm may be mounted in a sliding manner on a rail at the other end that is opposite the first pivot joint, by a second pivot joint. The rail extends in the longitudinal direction of the patient tunnel and renders it possible for the second pivot joint to be moved along the rail.

The pivot axis is a simple possibility of providing a defined pivoting movement about the vertical axis.

In one embodiment of the patient table, the patient table has a robot arm for positioning the bed surface, which is configured to position a patient on a bed surface of the patient table in an examination position. The robot arm directly produces a mechanical connection between the bed surface and the room in which the imaging apparatus is located (e.g., when the robot arm is anchored by one end directly to the floor or ceiling, or indirectly when a support apparatus is present). In this case, the positioning unit having the bed surface or the bed surface may be fixedly connected to the other, free end of the robot arm. For example, the connection between the robot arm and the positioning unit or bed surface is not only a gripper on the robot arm, but also a semi-permanent, rigid connection such as, for example, a screw connection or a positive-locking connecting element.

The robot arm is configured to pivot the bed surface about the vertical axis already described above. The robot arm may have a plurality of axes or degrees of freedom and supports the patient table or the support surface with the patient in order to be able to move the patient freely in the space, or at least to support the patient in the examination position in different orientations. In addition to the pivoting movement, a tilting movement about a longitudinal axis, for example, may also be provided. The robot arm may be firmly anchored to the floor of a treatment room, for example, so that the robot arm may receive and guide into the floor the forces that are introduced through the patient table or the bed surface into the robot arm in the different positions. In one embodiment, the patient table does not require any further support apparatus or suspension.

In one embodiment, a robot arm having sufficient degrees of freedom allows a pivoting movement about the vertical axis, which facilitates access to the patient. In addition, the robot arm may make further movements and inclinations for the bed surface that facilitate access.

In one embodiment of the patient table, the patient table has a bed surface for the patient. The bed surface is the surface on which the patient comes to lie directly. The bed surface also defines the orientation of the patient. The patient table has a tilting apparatus. The tilting apparatus is configured to orient the bed surface in a position tilted by a predetermined angle with respect to the horizontal about a longitudinal axis. In the case of an arrangement of the patient table in the position in accordance with the application in a magnetic resonance tomograph, an axis that extends essentially parallel to the longitudinal extension of the patient tunnel is considered to be a longitudinal axis. The tilting apparatus may have, for example, a physical axis or a pivot joint about which the support may be tilted, as well as a fixing element, such as, for example, a latch that fixes the set tilting angle. However, in one embodiment, the robot arm may perform the tilting movement in the case of a patient table having a robot arm.

Access may be improved (e.g., in the region of the rib cage and abdomen) by lateral tilting.

In one possible embodiment of the system, the support apparatus has a rail system having a curvature about the vertical axis. The curvature may be configured, for example, such that the rail forms a circular arc segment about the vertical axis. The system also has a guide element that corresponds to the rail system and engages with the rail system by a non-positive locking connection. For example, this may be a corresponding recess as a sliding bearing, in which the rail guides. Rollers or ball or roller bearings that engage with the rail and allow a particularly low-friction movement may also be provided. The patient table is configured to perform a pivoting movement about the vertical axis by moving along the rail system.

The rails render it possible for the patient table to be guided precisely and smoothly.

In one embodiment, the rail system is arranged on the patient table. The corresponding guide element is then arranged on the support apparatus (e.g., on the floor or a ceiling). Due to the limited space in the patient tunnel, an angle of pivoting movement is limited so that the rail system may also find space in a foot of the patient table.

Only a substantially smaller guide element is then required on the floor, which has space under the foot of the patient table or a positioning unit and thus does not obstruct an operating physician in the foot space.

A rail system on the floor or the ceiling may itself provide the support apparatus.

In one embodiment of the system, the rail system of the support apparatus is arranged above the imaging apparatus. In this case, the support apparatus may include a construction or a scaffold for receiving the weight of a patient table that is arranged in a suspended manner. In other words, the patient table is suspended so that the floor underneath remains free.

### BRIEF DESCRIPTION OF THE DRAWINGS

FIG. 1 shows a schematic illustration of an example system in accordance with the present embodiments;

FIG. 2 shows a schematic illustration of an example patient table in accordance with the present embodiments;

FIG. 3 shows a schematic illustration of an example system in accordance with the present embodiments;

FIG. 4 shows a schematic illustration of an example patient table in accordance with the present embodiments;

FIG. 5 shows a schematic plan view of an example embodiment of a patient table in accordance with the present embodiments;

FIG. 6 shows a schematic illustration of a detail of an example patient table in accordance with the present embodiments.

### DETAILED DESCRIPTION

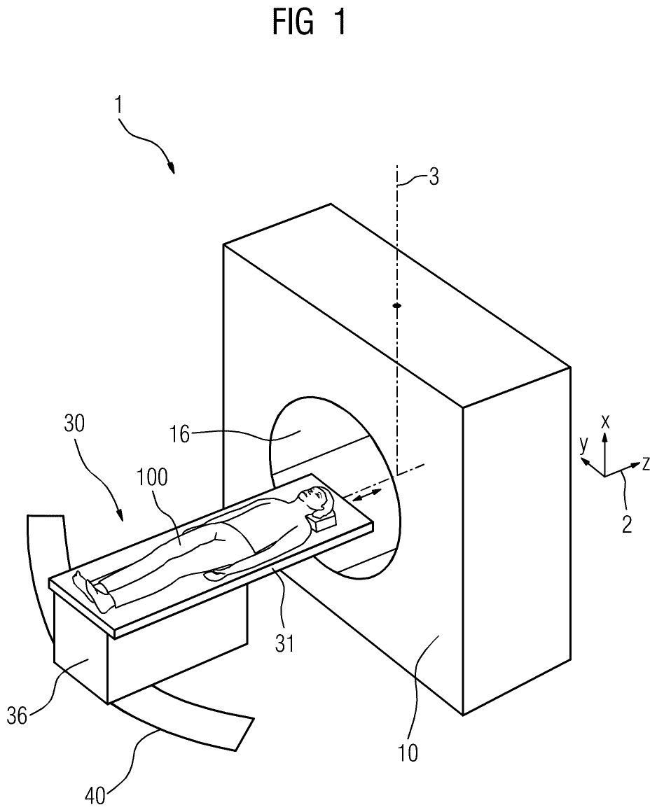

FIG. 1 illustrates an example embodiment of a system in accordance with the present embodiments. The system has a magnetic resonance tomograph 1 having a magnet unit 10. A patient tunnel 16 extends through the magnet unit 10 in a longitudinal direction 2 of the z-axis. The isocenter, with a location of a greatest magnetic field homogeneity, which may also be a center of symmetry of the magnetic field, is located in the patient tunnel 16 (e.g., in the center). The “field of view” extends around the isocenter, in which image acquisition may take place using the magnetic resonance tomograph 1. The vertical axis 3 extends perpendicularly along the direction of gravity (e.g., through the isocenter).

The positioning unit 36 of the patient table 30 is arranged outside the magnet unit 10, in FRONT of the MAGNET unit 10 in the longitudinal direction 2. The positioning unit 36 supports a bed surface 31 on which a patient 100 may be arranged for image acquisition. The positioning unit 36 is configured to move the bed surface 31 with the patient 100 into the patient tunnel 16. In one embodiment, the positioning unit 36 is configured to raise or lower the bed surface 31 in the direction of gravity or the x axis in order to simplify the positioning of the patient 100 on the bed surface 16.

For example, the positioning unit 36 of the embodiment that is illustrated in FIG. 1 is also configured to pivot the patient table 30 about the vertical axis 3. For this purpose, the embodiment of the system in FIG. 1 has a pivoting apparatus 40. In the illustration, the pivoting apparatus 40 is a rail or a rail system on the floor, in which the positioning unit 36 engages, so that the positioning unit 36 may be moved along the rail in a guided manner. The positioning unit 36 may, for example, have wheels on an underside of the positioning unit 36 that are oriented transversely in the y direction parallel to the rails and engage in the rails. However, in one embodiment, the wheels run on the ground, and only one coupling element engages in the rail and thus limits the movement of the positioning unit 36 on the wheels along the rail.

Conversely, a short rail may be arranged on the underside of the positioning unit 36. The short rail interacts with a coupling element on the floor. In this manner, an installation on the floor may be kept to a minimum, and there are no obstructive elements on the floor to the side of the patient table 30. Since the space for a pivoting movement of the patient table 30 about the vertical axis 3 is limited in the patient tunnel 16, a short rail in the positioning unit 36 may be sufficient.

In this case, the pivoting movement of the positioning unit 36 may be performed in a motorized manner or also manually.

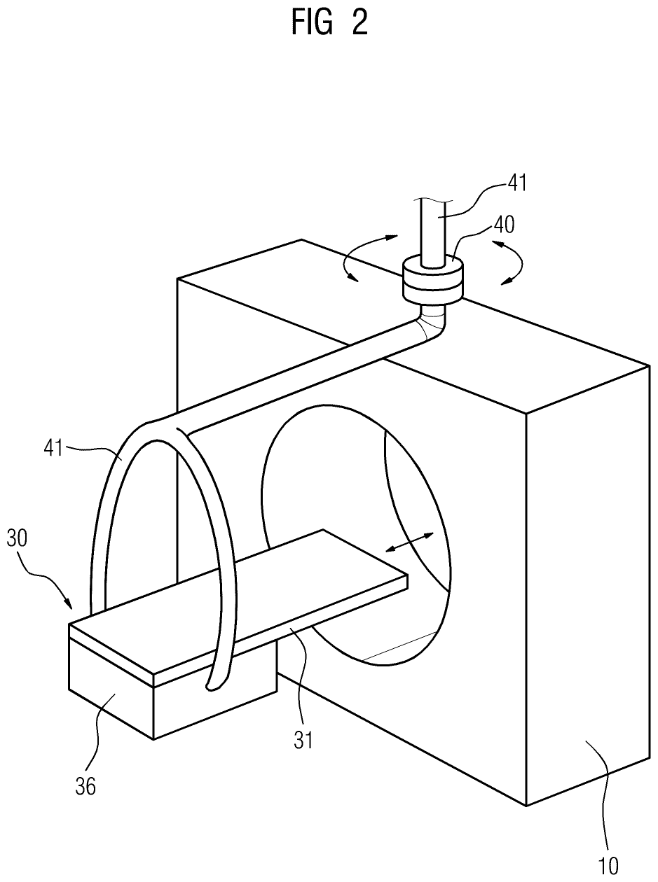

FIG. 2 illustrates a further possible embodiment of the system in accordance with the present embodiments. The essential difference from FIG. 1 is that the patient table 30 is not arranged standing on the floor, but suspended from the ceiling or a support apparatus. For this purpose, a suspension apparatus 41 is provided, fastened to the ceiling or support apparatus. The suspension apparatus 41 supports the patient table 30 having the positioning unit 36 and the bed surface 31. In this case, it is sufficient if the positioning unit 36 is configured to move the bed surface 31 in the longitudinal direction 3 into the patient tunnel.

The suspension apparatus 41 may be fastened to the ceiling or the support apparatus at an intersection of the vertical axis 3. A pivoting apparatus 40 (e.g., a pivot joint) is then arranged below along the vertical axis 3 in the suspension apparatus 41, so that the patient table 30 may be pivoted about the vertical axis 3. In this case, small deviations of the position of the pivot joint from the vertical axis 3 may also be provided.

In one embodiment, for example, the suspension apparatus 41 may not be fastened on both sides, but only with one arm on one side of the patient table 30 or the positioning unit 36 in order to facilitate access by the patient 100 to the bed surface 31.

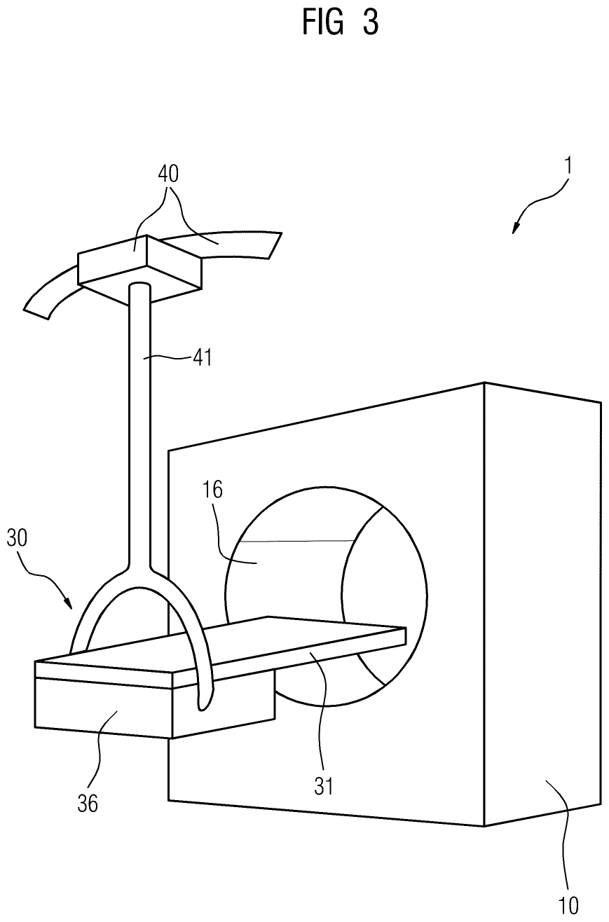

FIG. 3 illustrates a further embodiment of the system, in which a suspension apparatus 41 is combined with a rail system as a pivoting apparatus 40 on the ceiling or the support apparatus. As already described with regard to FIG. 1, the rail system in conjunction with coupling elements form the pivoting apparatus 40. The pivoting movement is effected by a movement of the suspension apparatus 41 along the rail system. For example, wheels on the suspension apparatus 41 may engage in an open U-shaped or inverted T-shaped rail.

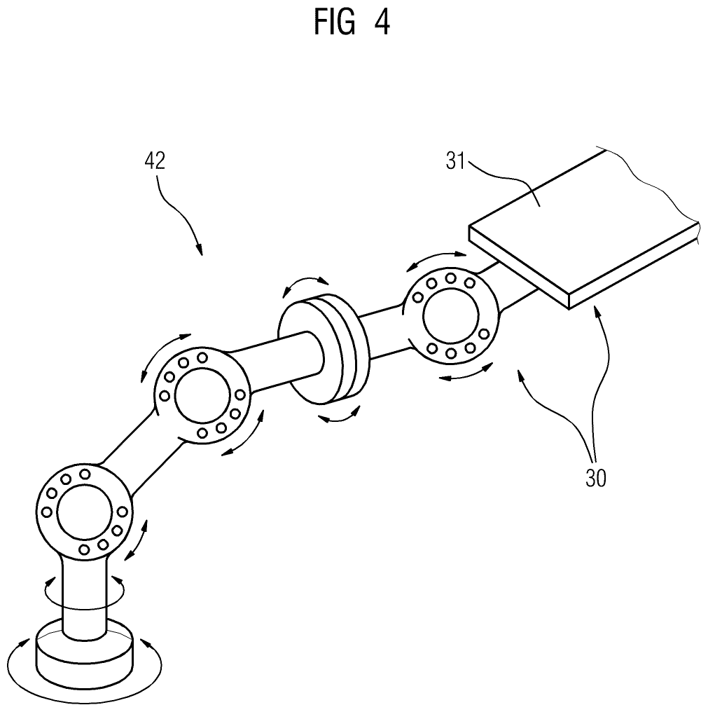

FIG. 4 illustrates a further embodiment of a system. In this embodiment, the pivoting apparatus 40 is provided by a robot arm 42. In this case, the patient table 30 is fastened to one end of the robot arm 42. The robot arm 42 is configured to provide the desired pivoting movement about the vertical axis 3 by a suitable movement about axes of rotation of the robot arm 42. In addition, the robot arm 42 may be configured to provide further movements by the combination of rotational movements about the axes of rotation (e.g., the movement with which the bed surface 31 with the patient 100 is introduced into the patient tunnel 16 in the longitudinal direction 2). Further, a movement of the patient table 30 along the vertical axis 3 may be provided in order to facilitate the positioning of the patient 100 on the patient table 30.

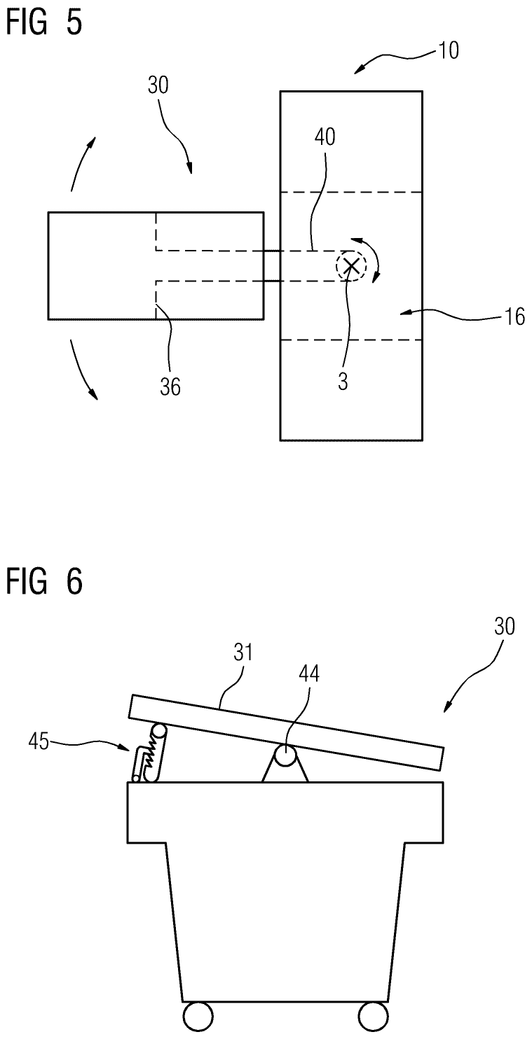

FIG. 5 illustrates a further possible embodiment. In this embodiment, the pivoting apparatus 40 is provided by a pivoting arm that connects a pivot joint at the location of the vertical axis 3 to the patient table 30 under the magnet unit 10, so that the pivoting arm and the pivot joint guide the patient table 30 in a pivoting movement about the vertical axis 3. The patient table 30 may have, for example, pivotable rollers or omnidirectional wheels on the underside in order to facilitate the pivoting movement. In one embodiment, the pivoting arm may be fastened rigidly, (e.g., with a fixed orientation) but in a detachable manner, to the patient table 30, so that the patient may be moved up to and coupled to the magnetic unit with the patient table 30.

For the intervention, the patient 100 is to be mounted at an ergonomic working height for the operating physician, which may be higher than the patient tunnel in the magnet unit 10. For this purpose, the magnet unit 10 may be mounted at a higher level, so that space is available underneath for the pivot joint and the pivot arm.

FIG. 6 illustrates a further embodiment of a patient table 30 in accordance with the present embodiments. In this patient table 30, the bed surface 31 is arranged on the positioning unit 36 so as to be tiltable on a tilting axis 44. In the case of image acquisition by the magnetic resonance tomograph 1, the tilting axis 44 may be arranged essentially parallel to the longitudinal direction 2, apart from the pivoting movement, so that the patient 100 on the bed surface 31 is inclined toward an operating physician next to the patient table 30 as a result of the tilting movement. In this case, the tilting angle may be fixed by a latch 45 that is arranged, for example, between the bed surface 31 and the positioning unit 36. A motorized adjusting facility may also be provided for this purpose.

Although the invention has been further illustrated and described in detail by the example embodiments, the invention is not limited in this regard by the disclosed examples, and other variations may be derived therefrom by the person skilled in the art without departing from the protective scope of the invention.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A patient table for a medical imaging apparatus, wherein the patient table is configured to support a patient in an examination position in the medical imaging apparatus for imaging, the patient table comprising:
a pivoting apparatus configured to pivot the patient about a vertical axis in the examination position.

2. The patient table of claim 1, wherein the medical imaging apparatus is a magnetic resonance tomograph.

3. The patient table of claim 1, further comprising a suspension apparatus configured for suspended fastening to a support apparatus.

4. The patient table of claim 3, wherein the pivoting apparatus is part of the suspension apparatus.

5. The patient table of claim 1, wherein the pivoting apparatus comprises a pivoting arm, and
wherein the pivoting arm is configured to guide the patient table during a pivoting movement about the vertical axis.

6. The patient table of claim 1, further comprising a robot arm for positioning, the robot arm being configured to:
position the patient on a bed surface of the patient table in the examination position; and
pivot the bed surface about the vertical axis.

7. The patient table of claim 1, further comprising:
a bed surface for the patient; and
a tilting apparatus about a longitudinal axis, the tilting apparatus being configured to orient the bed surface in a position tilted by a predetermined angle with respect to the horizontal.

8. The patient table of claim 4, further comprising:
a bed surface for the patient; and
a tilting apparatus about a longitudinal axis, the tilting apparatus being configured to orient the bed surface in a position tilted by a predetermined angle with respect to the horizontal.

9. The patient table of claim 5, further comprising:
a bed surface for the patient; and
a tilting apparatus about a longitudinal axis, the tilting apparatus being configured to orient the bed surface in a position tilted by a predetermined angle with respect to the horizontal.

10. The patient table of claim 6, further comprising:
a bed surface for the patient; and
a tilting apparatus about a longitudinal axis, the tilting apparatus being configured to orient the bed surface in a position tilted by a predetermined angle with respect to the horizontal.

11. A system comprising:
a patient table for a medical imaging apparatus, the patient table being configured to support a patient in an examination position in the medical imaging apparatus for imaging;
a support apparatus configured for arranging the patient table relative to the medical imaging apparatus; and
a pivoting apparatus configured to pivot the patient about a vertical axis in the examination position.

12. The system of claim 11, further comprising a rail system having a curvature about the vertical axis and a guide element for engaging in the rail system,
wherein the patient table is configured to perform a pivoting movement about the vertical axis by moving along the rail system.

13. The system of claim 12, wherein the rail system is arranged on the support apparatus, and the guide element is arranged on the patient table.

14. The system of claim 12, wherein the rail system is arranged on the patient table, and the guide element is arranged on the support apparatus.

15. The system of claim 11, wherein the support apparatus is arranged above the medical imaging apparatus.

16. The system of claim 13, wherein the support apparatus is arranged above the medical imaging apparatus.

17. The system of claim 14, wherein the support apparatus is arranged above the medical imaging apparatus.
